(19) United States
(12) Patent Application Publication
(10) Pub. No.: US 2019/0388144 A1
(43) Pub. Date: Dec. 26, 2019

(54) MEDICAL INSTRUMENT FOR ENDOSCOPIC MUCOSAL RESECTION AND ENDOSCOPIC SUBMUCOSAL DISSECTION

(71) Applicant: Wei Shen, Beijing (CN)

(72) Inventors: Jianguo Zhang, Beijing (CN); Wei Shen, Beijing (CN)

(73) Assignee: Wei Shen, Beijing (CN)

(21) Appl. No.: 16/017,585

(22) Filed: Jun. 25, 2018

(51) Int. Cl. A61B 18/14; A61B 10/04

(52) U.S. Cl. CPC A61B 18/1492; A61B 2017/00269; A61B 10/04

(57) ABSTRACT

A medical instrument for use in EMR and ESD procedures includes a proximal handle having an electrical connection plug, a slidable handle portion, and a rotatable handle portion. The medical instrument includes a sheath extending from the proximal handle, the sheath comprising a lumen and a distal end away from the proximal handle. A cutting assembly extends through the sheath and comprises a wire and a working end. The cutting assembly and sheath are moveable relative to each other to cover and reveal the working end such that the working end has a retracted position and a working position. The working end comprises a flexible knife comprising a distal end, a proximal end, and an insulated portion. An insulated spear-shaped tip is provided at the distal end of the flexible knife, the insulated spear-shaped tip preventing cutting of a layer other than a mucosal layer or a submucosal layer.

### TECHNICAL FIELD

The invention relates generally to medical instruments used for endoscopic mucosal resection and submucosal dissection.

### BACKGROUND

One of the most common forms of cancer is colon cancer. Most cases begin with formation of small, noncancerous clumps of cells called lesions or polyps in the large intestine. The polyps may produce few, if any, symptoms, but the polyps can become colon cancer over time. Physicians recommend screening and polyp removal before the polyps turn into cancer.

Colonoscopy may be used for diagnosis and removal of the polyps. The procedure uses a long, flexible, slender tube attached with a camera to view the colon and rectum. If suspicious areas are found, the physician may pass surgical tools through the tube. The surgical tools may be used to take tissue samples and remove polyps.

When the entire polyp is removed, there is a lower risk of reoccurrence of polyps in the removal area. Removing each polyp as a whole, or “en bloc”, allows for further study of the polyp and is useful for diagnostic and treatment purposes. In the removal procedure, a surgical tool would be used to cut into the mucosal layer and the submucosal layer to remove the polyp. Complications arise when cutting beyond the submucosal layer. For example, the physician may cut into the muscular layer and may perforate the colon, there may be excess bleeding or infection, and there may be increased recovery time.

### SUMMARY

Removal of the entire lesion or polyp, while keeping the lesion or polyp intact, is difficult when using traditional, rigid surgical tools that require extensive training before use and have a complicated method of use. The present invention provides a medical instrument with a flexible, rotatable knife that uses an electric current to cut into the tissue layers and a simple removal process for removal of lesions or polyps. The flexible knife has a spear-shaped insulated tip that guides the flexible knife and prevents the knife from cutting into the muscular layer. The present invention may be used for en bloc resection or dissection of lesions or polyps and provides an easy, effective procedure.

The flexible knife may extend from the flexible tubing or sheath to expose different lengths of the knife. For example, the flexible knife may extend a further distance from a distal end of the sheath and thus extend a further distance from the distal end of the endoscope than traditional knives. For example, a flexible knife according to the present invention may extend about 2 cm from the distal end of the sheath, which is a length greater than the 1-2 mm length offered by traditional knives. This allows the sheath to avoid contacting the lesion or polyp while the knife is used to cut the lesion or polyp, thereby maintaining the integrity of the lesion or polyp. This also allows for better view of the procedure, as a camera in an endoscope through which the instrument is received may not be impeded by blood flow resulting from cutting the lesion or polyp.

Furthermore, a procedure using a flexible knife of the present invention has a shorter duration than a similar procedure using a traditional knife. When using a traditional knife, the tip of the knife and the end of the endoscope are close in distance to the polyp or lesion to be removed. While cutting the polyp or lesion, the process must be stopped in order to cauterize the wound and halt excess bleeding which impedes the view of the endoscope camera. In a process according to the present invention, the endoscope camera is located a further distance away from the polyp or lesion to be removed. In addition, the sweeping motion of cutting the polyp or lesion with the electric current produces less excess bleeding and thus does not require stopping of the process to cauterize wound as frequently. Because there is less stopping to cauterize, removal of a polyp or lesion using a procedure according to the present invention has a shorter duration than similar processes using traditional knives. Additionally, the flexible knife of the medical instrument is easier to use and has less training time than the traditional short, rigid-tipped surgical tools.

Typically, traditional knives have tips that are short and hard, have a transparent hood, and have less rotation and length adjustment. The traditional knives are used to carve out the lesion or polyp and are restricted by a short distance and limited vision when cutting. The length of the knife is not adjustable for cutting at different depths. While cutting, the knife has to be very close to the lesion or polyp, with the nose of the knife pointed downward and then rising to carve out the lesion or polyp. The integrity of the lesion or polyp may be damaged if the end of the knife and the cover of the knife directly touch the lesion or polyp. The knife does not exhibit flexibility when cutting. The angle of approach may not be ideal with the traditional knife, as the end of the knife is rigid and cannot bend or contour over the surface of the gastrointestinal (GI) tract. This may lead to cutting through the tissue layers and into the muscular layer.

The present invention may be used for endoscopic mucosal resection (EMR) or endoscopic submucosal dissection (ESD). EMR and ESD are two procedures conducted to remove lesions or polyps in the GI tract. The EMR procedure takes less time and is a less complicated procedure than the ESD procedure. However, EMR does not achieve en bloc removal of lesions or polyps, does not allow for precise histological stage, and there is a high risk of reoccurrence of polyps or lesions in the removal area. The medical instrument of the present invention may be used for EMR and ESD. The sheath of the medical instrument may further be configured to operate within a working channel in an endoscope.

In an aspect of the invention, a medical instrument comprises a proximal handle comprising an electrical connection plug, a slidable handle portion, and a rotatable handle portion. A sheath extends from the proximal handle, the sheath comprising a lumen and a distal end away from the proximal handle. A cutting assembly extends through the sheath, the cutting assembly comprising a wire and a working end, the cutting assembly and sheath moveable relative to each other to cover and reveal the working end such that the working end has a retracted position and a working position. The working end comprises a flexible knife comprising a distal end, a proximal end, and an insulated portion. An insulated spear-shaped tip is provided at the distal end of the flexible knife, and the insulated spear-shaped tip preventing cutting of a layer other than a mucosal layer or a submucosal layer. The insulated spear-shaped tip further acts as a pivot point when the distal end of the flexible knife is pushed down, which allows the cutting edge to be rotated about that pivot point.

The slidable handle portion can be manipulated by a user to move the working end forward and backward between the retracted position and working position. The rotatable handle portion can be manipulated by the user to rotate the cutting assembly. The rotatable handle may also be manipulated to stay in a locked position so that no rotation occurs. Alternatively, the sheath can be operable by the proximal handle to cover and reveal the working end of the cutting assembly from the distal end of the sheath.

The flexible knife of the invention uses electrical current to cut tissue. In particular, part of the flexible knife may be exposed in order to use the electrical current for cutting. The part of the flexible knife that is not used for cutting may be insulated. The electrical connection plug in the proximal handle is configured to connect to an electric power source to supply the electrical current. The wire extending through the sheath may be insulated. The proximal end of the flexible knife connects to the wire. In an embodiment, the flexible knife comprises a wire loop. The insulated portion may comprise a portion of the flexible knife from the insulated spear-shaped tip to the proximal end of the flexible knife.

Looking at the knife when extended from the sheath, one side of the knife may be exposed and used for electrical cutting action. The remaining portion of the knife may be insulated from the spear-shaped tip on the distal end of the knife to the sheath. The insulated part of the knife clings to the tissue while the knife is electrically cutting, helping to guide and contour the knife over the terrain and prevent cutting into other layers.

The medical instrument of the present invention may be used to electrically cut tissue, such as a polyp or lesion. Dissection or resection of the lesion or polyp involves cutting the lesion or polyp out of the mucosal and/or submucosal layer. However, cutting too deep may result in cutting the muscular layer and perforating the intestinal wall. The insulated spear-shaped tip of the present invention guides the flexible knife into the lesion or polyp and prevents cutting of a layer other than a mucosal layer or a submucosal layer.

Further, the present invention uses a sweeping cutting motion instead of cutting away a polyp or lesion in a carving manner, such as used by traditional knives. For example, electrically cutting is in a sweeping motion may entail cutting from one side of the lesion or polyp to the opposite side of the lesion or polyp.

Other aspects and advantages of the invention will be apparent upon consideration of the following detailed description thereof.

### BRIEF DESCRIPTION OF THE DRAWINGS

FIG. 1 is a perspective view of the medical instrument with flexible knife retracted.

FIG. 2 is a perspective view of the medical instrument received within an endoscope.

FIG. 3 is a view of the medical instrument proximal handle.

FIG. 4 is a cutaway view of the stabilizing collar where the sheath and proximal handle intersect.

FIG. 5 is a view of a distal end of the sheath with the flexible knife retracted.

FIG. 6 is a cutaway view of the proximal handle.

FIG. 7 is a side elevation of the medical instrument showing manipulation of the proximal handle to deploy the flexible knife.

FIG. 8 is an enlarged elevation of the flexible knife, showing the flexible knife slightly deployed from the sheath.

FIG. 9 is a side elevation of the medical instrument showing manipulation of the proximal handle to deploy the flexible knife.

FIG. 10 is an enlarged elevation of the flexible knife, showing the flexible knife partially deployed from the sheath.

FIG. 11 is a side elevation of the medical instrument showing manipulation of the proximal handle to fully deploy the flexible knife.

FIG. 12 is an enlarged elevation of the flexible knife, showing the flexible knife fully deployed from the sheath.

FIG. 13 is a perspective view of the medical instrument used in an endoscope, showing the rotation and flexibility of the extended flexible knife.

FIG. 14 is a perspective view of the medical instrument in an endoscope, showing the knife plunging into an object.

FIG. 15 is an enlarged view of the insulated spear-shaped tip.

FIG. 16 is a view of the flexible knife conforming to the terrain at a first position.

FIG. 17 is a view of the flexible knife conforming to the terrain at a second position.

FIG. 18 is a view of the flexible knife conforming to the terrain at a third position.

FIG. 19 is a view of the marked lesion or polyp, with the injector adjacent.

FIG. 20 is a view of the marked lesion or polyp, showing the injector enlarging the submucosa.

FIG. 21 is a perspective view of the flexible knife slightly deployed, spaced from the inflated submucosa.

FIG. 22 is a perspective view of the flexible knife slightly deployed, with the flexible knife making the initial cut parallel to the gastrointestinal wall.

FIG. 23 shows the flexible knife being further deployed.

FIG. 24 shows the flexible knife inserted deeper into the submucosa.

FIG. 25 shows the flexible knife cutting from right to left.

FIG. 26 shows the flexible knife fully deployed.

FIG. 27 shows the flexible knife cutting from right to left and reaching the distal edge of a lesion or polyp.

FIG. 28 shows the flexible knife cutting around the perimeter of the lesion or polyp.

FIG. 29 shows the removal of the lesion or polyp.

### DETAILED DESCRIPTION

The present invention provides a medical instrument having a flexible knife that is rotatable, extends from a sheath, uses electric current to cut, and is operable by a proximal handle. A distal end of the flexible knife has an insulated spear-shaped tip. The tip helps to guide the flexible knife into a lesion or polyp, contour to terrain, and prevent the flexible knife from cutting into layers that are not the mucosal layer or submucosal layer, e.g. the muscular layer. The flexible knife may comprise a wire loop. Looking at the knife when extended from the sheath, one side of the knife may be exposed and used for electrical cutting action. The remaining portion of the knife may be insulated from the spear shaped tip on the distal end of the knife to the sheath. The insulated part of the knife clings to the tissue while the knife is electrically cutting.

The present invention has greater flexibility and rotation capabilities than a knife having a short, rigid tip. For instance, the flexible knife can extend from the sheath and embody different lengths. The knife may be fully retracted in the sheath, may extend partially out of the sheath, or may extend fully out of the sheath. The knife may comprise a flexible, electrically conductive wire. The wire may be a braided wire.

Traditional knives require use of transparent hoods or caps during EMR or ESD procedures. The cap is used to maintain visualization when using a traditional knife during an EMR or ESD procedure, as the cap keeps the resected flap of mucosa off of the endoscope lens. The present invention does not require a transparent hood. The adjustable knife length of the present invention allows extension of the knife from the sheath and provides options for length of the knife during use. As such, the dissection or resection may be carried out at a distance from the endoscope.

Traditional knives also have knife tips that are difficult to insert into the submucosa layer. Insertion difficulty of the knife tip on a traditional knife may lead to using excess force and cutting deeper than the submucosa layer. In the present invention, the insulated spear-shaped tip experiences less difficulty plunging into the submucosa and prevents the knife from cutting into the muscular layer.

Furthermore, when using traditional knives, any cutting direction adjustment is carried out by endoscopy. The present invention allows for adjustment of the cutting direction by using endoscopy or by adjusting the cutting assembly by manipulation of the proximal handle. The cutting assembly is flexible and rotatable. Further, the present invention has options for the length of extension or protrusion of the cutting assembly from the sheath, thereby allowing for use of different lengths of the exposed knife for electrical cutting.

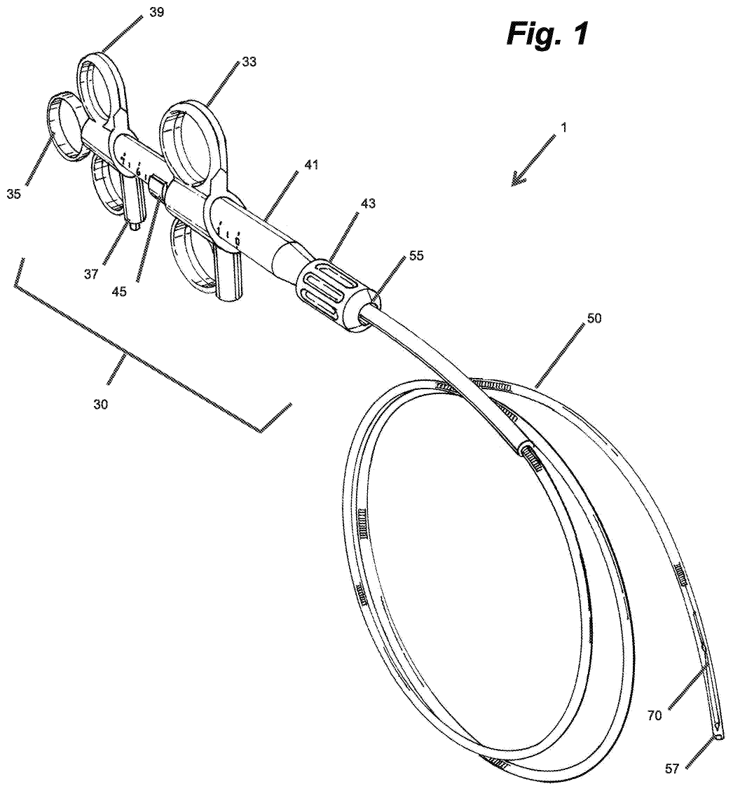

FIG. 1 shows the medical instrument 1 of the present invention with the cutting assembly 70 in a retracted position. The instrument 1 comprises a proximal handle 30, a sheath 50, and a cutting assembly 70. The proximal handle 30 may have an electrical connection plug 37, a rotatable handle portion 35, a slidable handle portion 39, a second slidable handle portion 33, a stabilizing collar 43, a body 41, and a setting clip 45. The proximal handle 30 may comprise any suitable material, such as metal or plastic. The sheath 50 has a proximal end 55 and a distal end 57. The sheath 50 may comprise any suitable material that is flexible, such as plastic tubing. The cutting assembly 70 may comprise any suitable electrically conductive material, such as a metal (e.g. copper, silver, gold, aluminum, zinc).

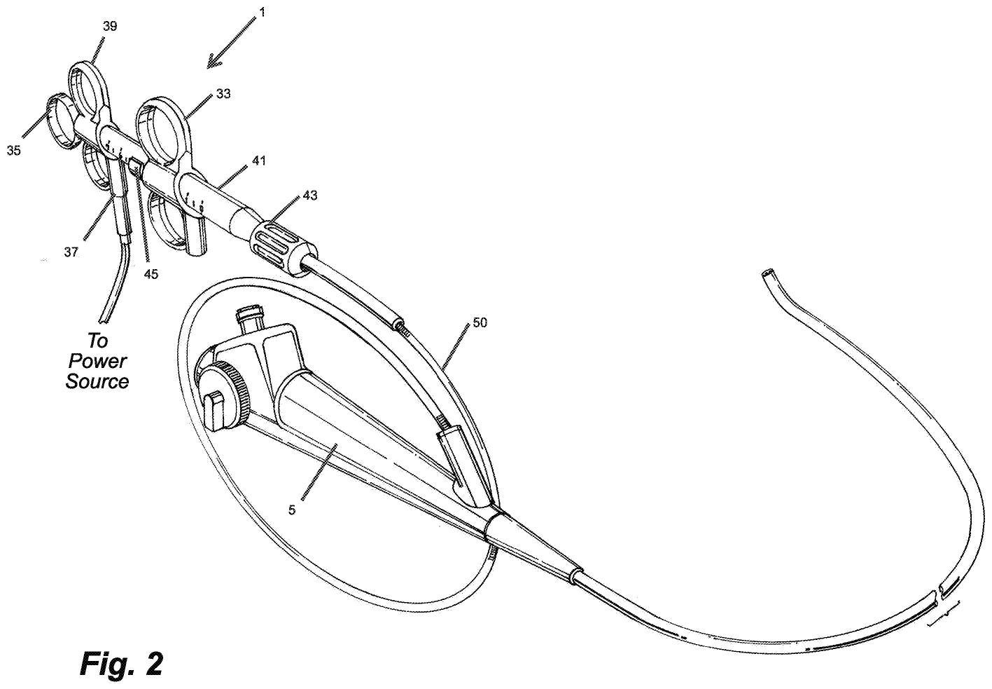

One aspect of the medical instrument of the present invention is that the instrument 1 may be received within an endoscope 5, such as shown in FIG. 2. The endoscope 5 may be used for a procedure such as a colonoscopy and may provide several options for working channels of the endoscope, such as a camera, lighting, suction, and water. FIG. 2 also depicts the electrical connection plug 37 which is configured to be connected to a power source. By connecting to an electric power source, electricity is provided to the cutting assembly and allows cutting of tissue, such as polyps or lesions in the mucosal or submucosal layers.

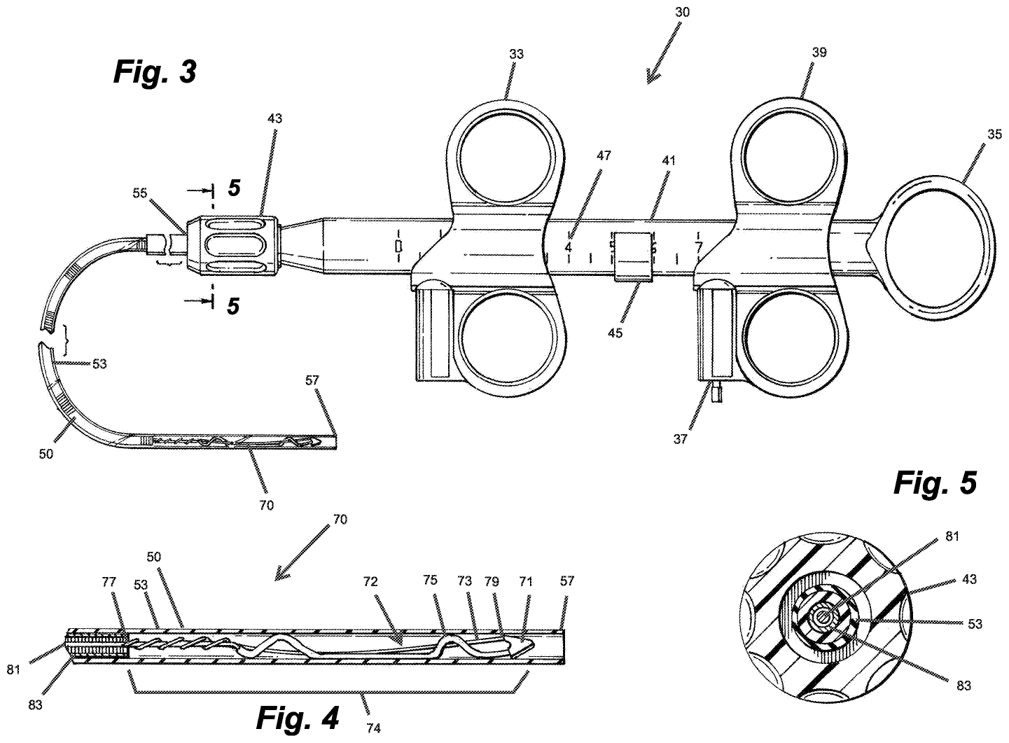

FIG. 3 shows the proximal handle 30 and distal end 57 of the sheath 50 with the cutting assembly 70 in a retracted position. Breaks are shown in the lumen 53 of sheath 50 between the proximal end 55 and distal end 57 of the sheath 50. FIG. 4 is an enlarged view of the cutting assembly 70 and shows the connection between the working end 74 and the wire 81 of the cutting assembly 70. The working end 74 comprises a flexible knife 72 and an insulated spear-shaped tip 71. FIG. 4 shows an insulated portion 75 of the flexible knife 72 and an exposed portion 73 of the flexible knife 72, which is used for electrical cutting. The insulated portion 75 may be covered with any suitable insulating material, such as plastic or rubber. The insulated spear-shaped tip 71 may also be formed from any suitable insulating material, such as plastic or rubber. Insulation on the insulated portion 75 of the flexible knife and the insulated spear-shaped tip 71 provides additional safety during use and prevents added injury to the patient. The cutting assembly 70 is shown in a retracted position in FIG. 4, and the cutting assembly 70 does not extend from the distal end 57 of the sheath 50 in a retracted position.

FIG. 5 shows a cutaway view along line 5-5 of FIG. 3 to show the stabilizing collar 43 where the lumen 53 of the sheath 50 and proximal handle 30 meet. The stabilizing collar 43 may be held by a user while the user rotates the rotatable handle portion 35 to rotate the cutting assembly 70. Arrangement of the wire 81, lumen 53 of the sheath 50, and metal lumen 83 within the stabilizing collar 43 are shown in FIG. 5. A user may also hold the stabilizing collar 43 while the rotatable handle portion 35 is not being rotated, thus maintaining a locked position where no rotation occurs.

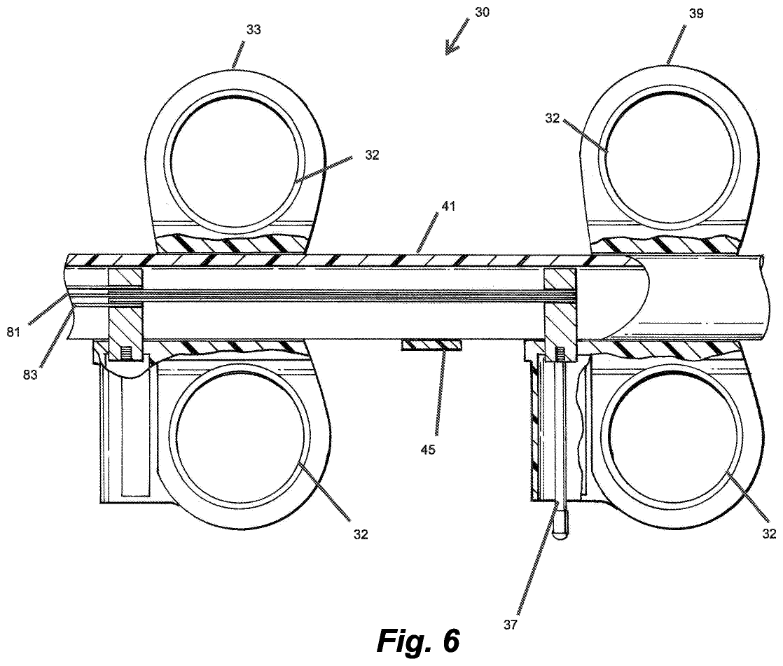

FIG. 6 shows a partial cutaway view to display the inner workings of the proximal handle 30. The slidable handle portion 39 and second slidable handle portion 33 may be configured to comfortably support a user's grip and have finger holds 32. The slidable handle portion 39 and second slidable handle portion 33 may slide along a body 41 of the proximal handle 30 in a forward and backward direction. Within the body 41, the wire 81 of the cutting assembly 70 is attached to the slidable handle portion 39 so that the wire 81 is movable by moving the slidable handle portion 39. The slidable handle portion 39 also includes an electrical connection plug 37, which is configured to accept an electrical current from an electric power source and provide an electrical current to the wire 81. The wire 81 comprises an electrically conductive material and conducts the electricity to the flexible knife 72 of the cutting assembly 70. A second slidable handle portion 33 may be connected to a metal lumen 83 so that forward or backward movement of the second slidable handle portion 33 moves the metal lumen 83 forward or backward in the sheath 50. The metal lumen 83 may be arranged inside the lumen 53 of the sheath 50 and may further protect the wire 81 when the wire 81 is arranged inside the metal lumen 83.

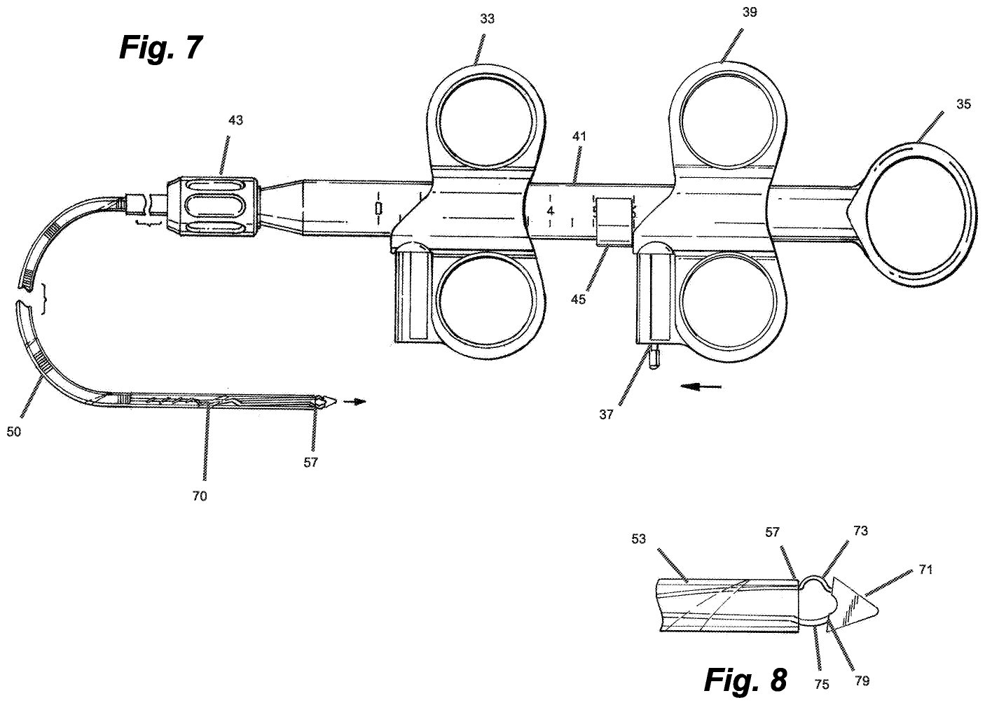

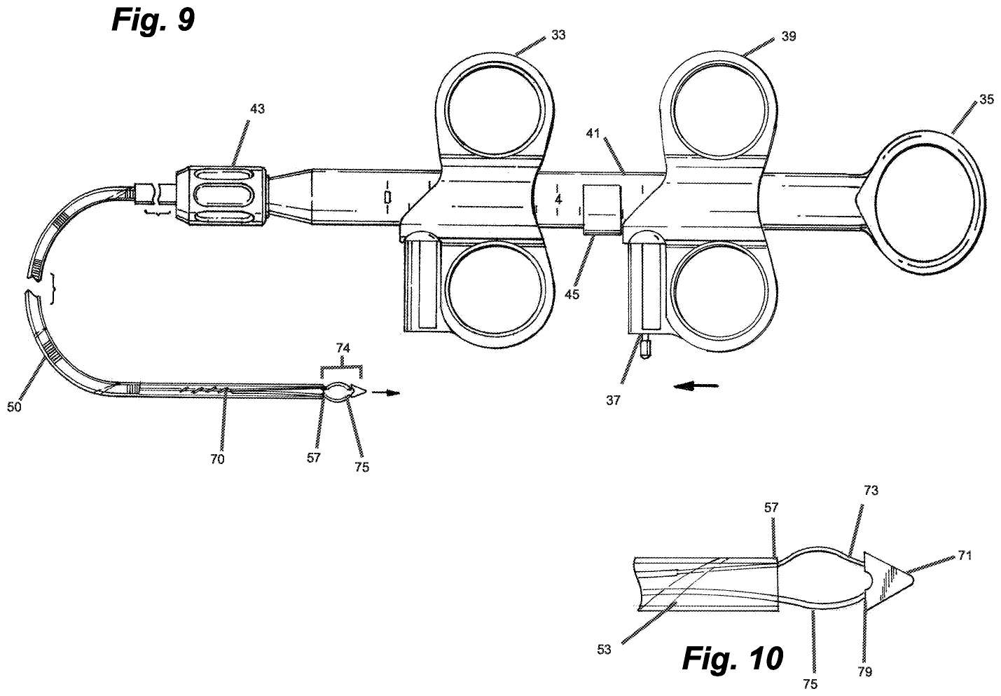

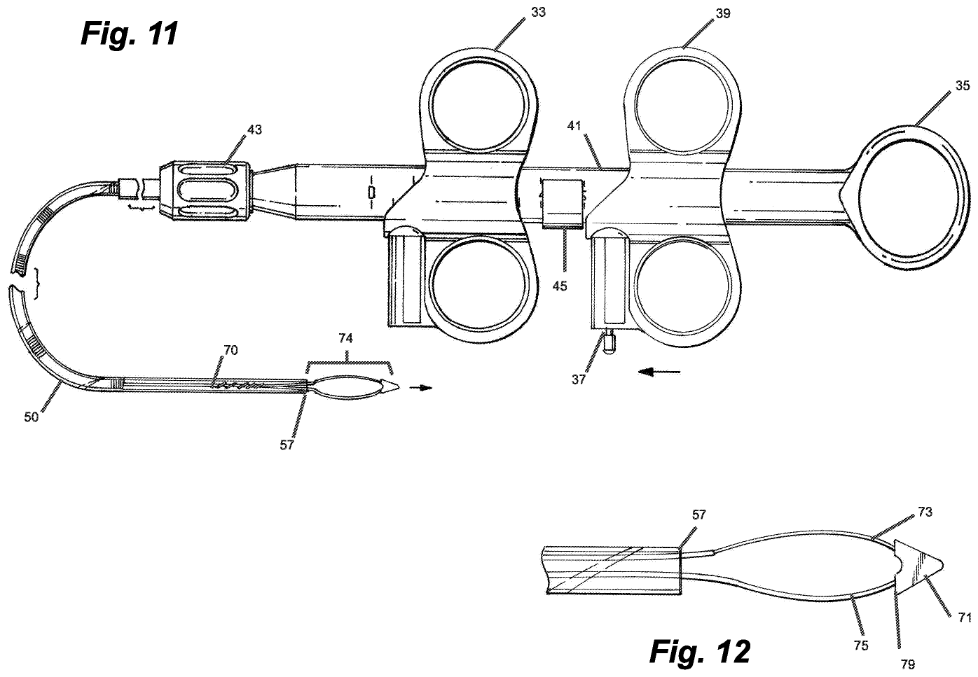

FIGS. 7, 8, 9, 10, 11, and 12 depict manipulation of the proximal handle 30 to deploy a working end 74 of the cutting assembly 70 from the distal end 57 of the sheath 50. FIG. 7 shows the slidable handle portion 39 moving in a forward direction to partially deploy or extend the cutting assembly 70. FIG. 8 shows the result of the proximal handle manipulations of FIG. 7, namely that the cutting assembly 70 is slightly deployed from the distal end 57 of the lumen 53 of the sheath 50. FIG. 9 shows the slidable handle portion 39 moving in a further forward direction to further deploy or extend the working end 74 of the cutting assembly 70. FIG. 10 shows the result of the proximal handle manipulations of FIG. 9, namely that the cutting assembly 70 is further deployed from the distal end 57 of the lumen 53 of the sheath 50. FIG. 11 shows the slidable handle portion 39 moving in a further forward direction to fully deploy or extend the working end 74 of the cutting assembly 70. FIG. 12 shows the result of the proximal handle manipulations of FIG. 11, namely that the cutting assembly 70 is fully deployed from the distal end 57 of the lumen 53 of the sheath 50.

A body 41 of the proximal handle 30 may further comprise graduated markings or gradient markers 47. The gradient markers 47 may correspond to a length of the wire 81 for extending or retracting the working end 74 of the cutting assembly 70 from the sheath 50.

Optionally, a setting clip 45 may be used to specify a distance for moving the slidable handle portion 39 and the second slidable handle portion 33. The setting clip 45 may be arranged on the body 41 of the proximal handle 30 at a position marked by the gradient markers 47. The setting clip 45 may be arranged on the body 41 of the proximal handle 30 at a position between the slidable handle portion 39 and the second slidable handle portion 33.

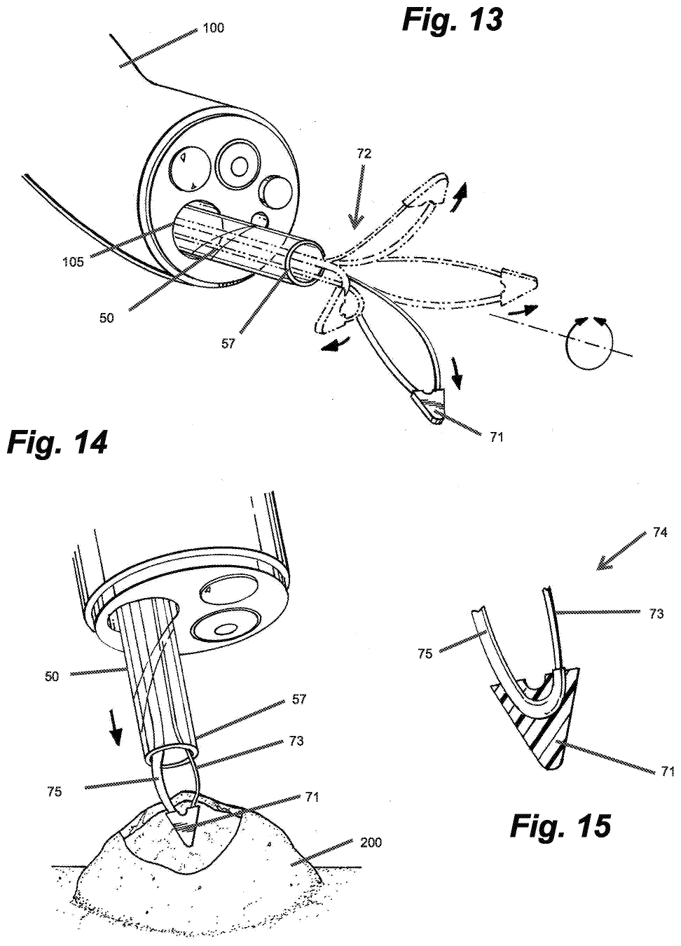

FIGS. 13 and 14 show the medical instrument of the present invention arranged in a working channel 105 of an endoscope 100. FIG. 13 depicts the flexibility and rotation features of the flexible knife 72 according to the present invention, as shown by the various positions of the extended flexible knife 72. FIG. 14 shows the working end 74 of the cutting assembly extending from the distal end 57 of the sheath 50 with the insulated spear-shaped tip 71 plunging into an object 200, such as a lesion or polyp. FIG. 15 shows an enlarged view of the working end 74 of the cutting assembly, namely showing the insulated spear-shaped tip 71 and the flexible knife, the flexible knife comprising an insulated portion 75 and an exposed portion 73.

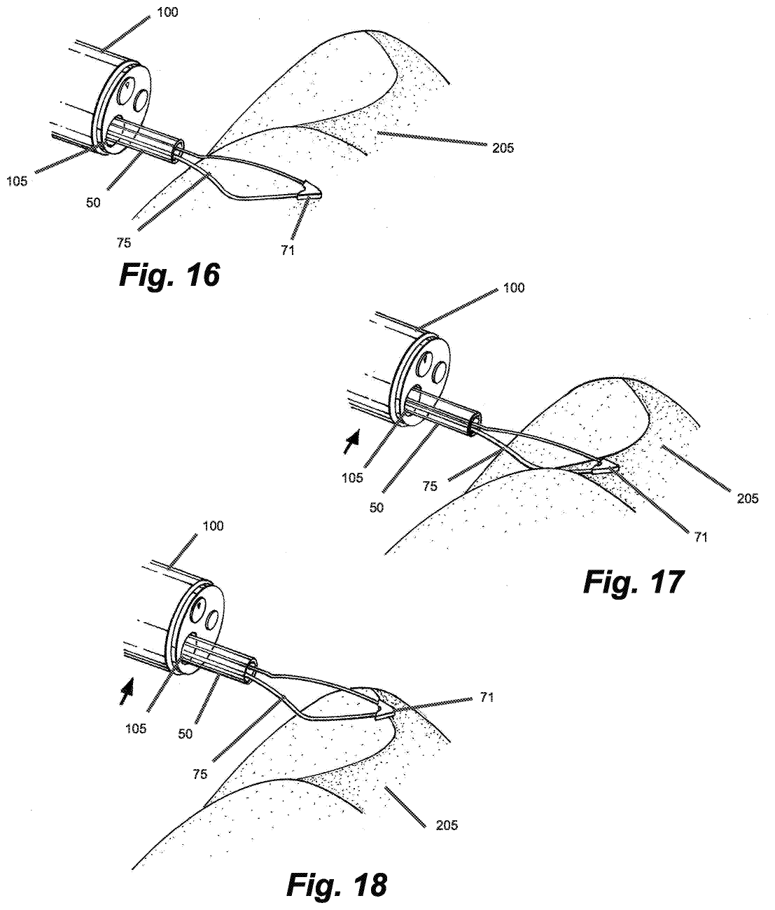

FIGS. 16, 17, and 18 show the flexibility of the present invention and the ability of the cutting assembly to contour to the terrain of an object without getting caught on or cutting into the object, such as a gastrointestinal wall. FIG. 16 shows the insulated portion 75 of the flexible knife gripping to the tissue and conforming to the terrain of an object 205 at a first position. FIG. 17 shows progression of the instrument moving in one direction and continuing to conform to the terrain of the object 205 at a second position. FIG. 18 shows the progression of the instrument continuing to move in the same direction and maintaining to conform to the terrain of the object 205 at a third position.

The procedural steps for EMR and ESD are similar. The lesion or polyp is marked, fluid is injected into the submucosa to elevate the lesion or polyp, the mucosa surrounding the lesion or polyp is cut, and the submucosa beneath the lesion or polyp is dissected. Marking may be done using cautery or small electrosurgical burns (e.g. argon plasma coagulator) to mark the periphery of the lesion or polyp. A saline solution that includes a dye (e.g. indigo carmine, methylene blue) is typically used for injection.

The core step of EMR or ESD is resection or dissection of the lesion or polyp to produce a sample to be used for diagnosis and evaluation. En bloc resection is more desirable than piecemeal resection, as it allows for more accurate assessment and appropriateness of the therapy. As such, ESD is preferably performed, as ESD provides a sample of the lesion or polyp en bloc. The collected sample may then undergo strict and precise pathological diagnosis and evaluation. Complications may arise from the procedure, such as perforation, bleeding, stenosis, etc., which may lead to increased recovery time and long-term follow-up. Steps for the resection or dissection procedure include marking the lesion or polyp, injection, incision, dissection, sample collection of the lesion or polyp, and wound management.

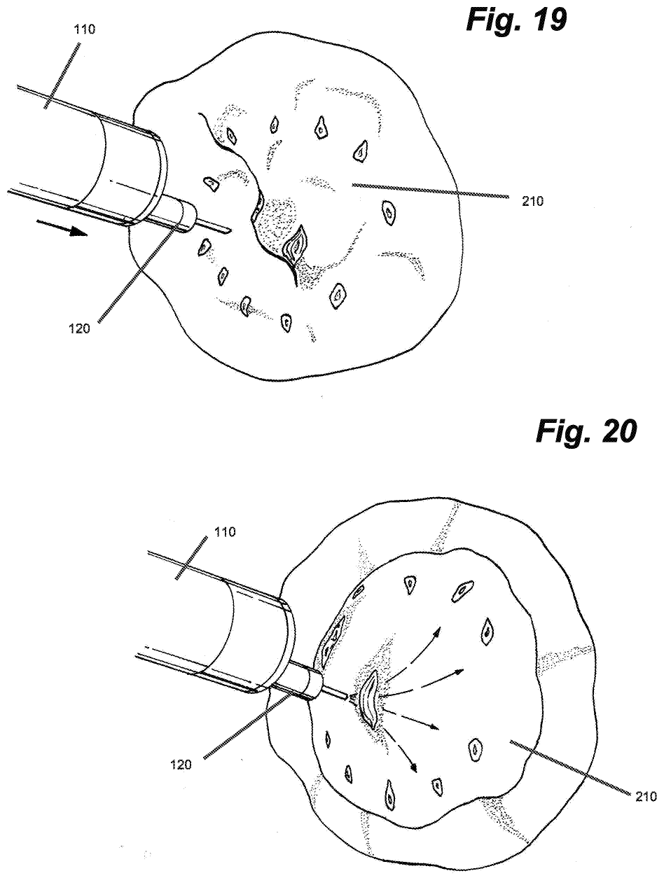

FIGS. 19-29 depict use of an instrument according to the present invention in an ESD or EMR procedure. FIGS. 19 and 20 show initial steps where the lesion or polyp is marked and injected. In FIG. 19, the electrosurgery markings around the periphery of the lesion or polyp 210 are shown. The injector 120 is adjacent to the marked lesion or polyp 210 and extends from the endoscope 110. The injector 120 then injects a solution, e.g. a saline solution preferably containing a dye such as methylene blue, to elevate or raise the lesion or polyp for ease of dissection. The injection step is shown in FIG. 20.

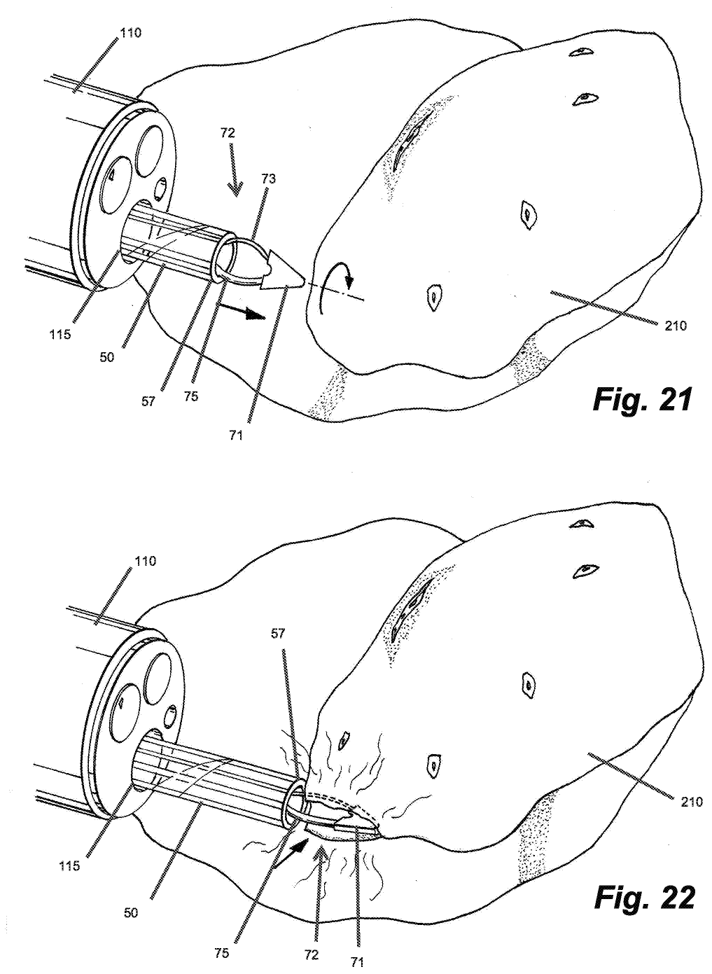

The resection or dissection steps are shown in FIGS. 21-29. For the resection or dissection step, the injector 120 in a working channel 115 of the endoscope 110 is replaced with the instrument of the present invention. Optionally, the injector may be arranged in a separate working channel of the endoscope and retracted during the resection or dissection step.

FIG. 21 shows the initial approach for the resection or dissection. The insulated spear-shaped tip 71, exposed portion 73, and insulated portion 75 of a flexible knife 72 of the present invention are slightly deployed. The flexible knife 72 is spaced from the lesion or polyp 210 and may be rotated or further extended from the sheath 50 before incision. The endoscope 110 is arranged parallel to the gastrointestinal wall and the base of the lesion or polyp 210.

FIG. 22 shows the flexible knife 72 making the initial cut into the lesion or polyp 210. The instrument may be arranged at any suitable angle, but is preferably parallel to the gastrointestinal wall while cutting to minimize added injury to the patient and reduce risk of perforation from cutting. The instrument is used to make a small initial incision on the lesion or polyp 210, such as on a right side of the lesion or polyp, using electric current.

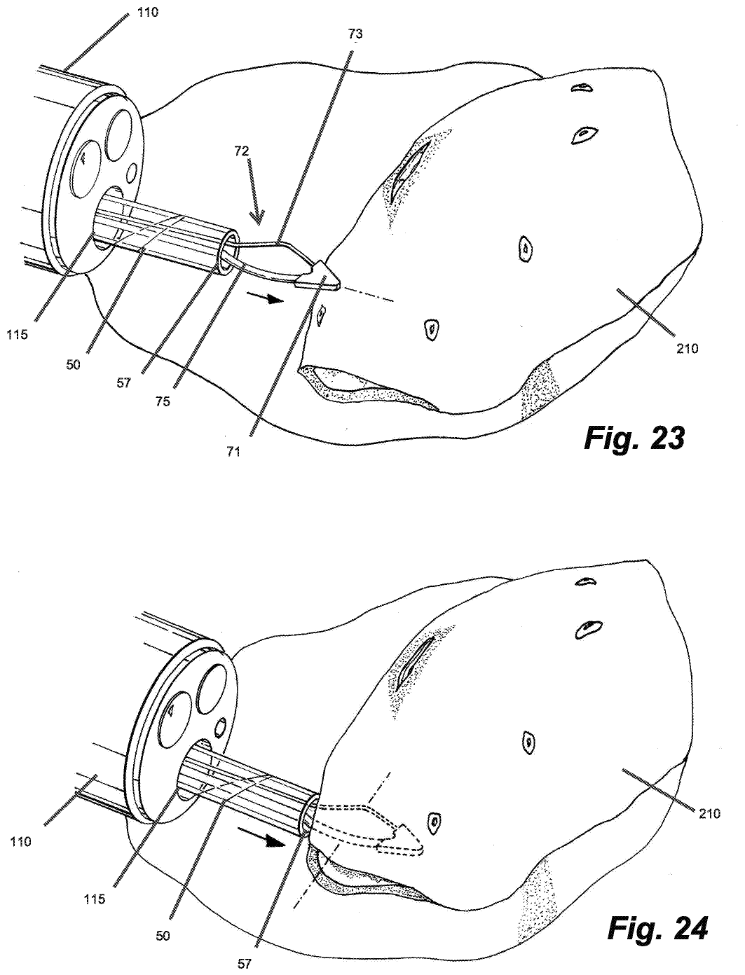

FIG. 23 shows the flexible knife 72 extending a further distance from the distal end 57 of the sheath 50. This partially deploys the flexible knife 72 and exposes a larger section of the flexible knife 72 for use when cutting into the lesion or polyp 210. The flexible knife is then inserted into the lesion or polyp at the incision point, such as on the right side of the lesion or polyp, and cuts in a sweeping motion from right to left. FIG. 24 shows the flexible knife inserted deeper into the lesion or polyp 210 and cutting in the sweeping motion from the right side of the lesion or polyp to the left side of the lesion or polyp. The endoscope and flexible knife preferably remain parallel to the gastrointestinal wall and base of the lesion or polyp.

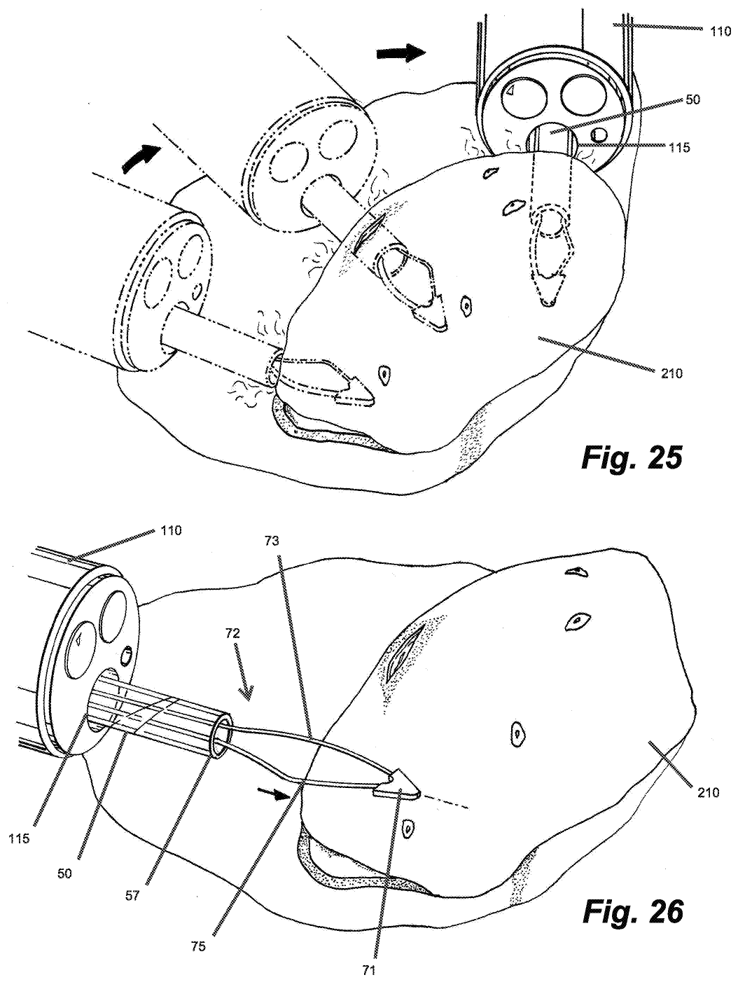

FIG. 25 shows the flexible knife when inserted further into the lesion or polyp during dissection or resection. The knife starts at a first position on the right side of the lesion or polyp and moves leftward in a fluid motion. The knife remains parallel to the gastrointestinal wall and cuts in a sweeping motion from the right side to the left side of the lesion or polyp 210. The endoscope remains distanced from the flap of the lesion or polyp that has already been cut, which is due to the ability of the present invention to extend the flexible knife at various distances from the distal end of the sheath 50. Extension of the flexible knife allows the present invention to avoid endoscope camera impedance associated with excess bleeding from the procedure as well as endoscope camera impedance from the flap of the lesion or polyp that has already been cut.

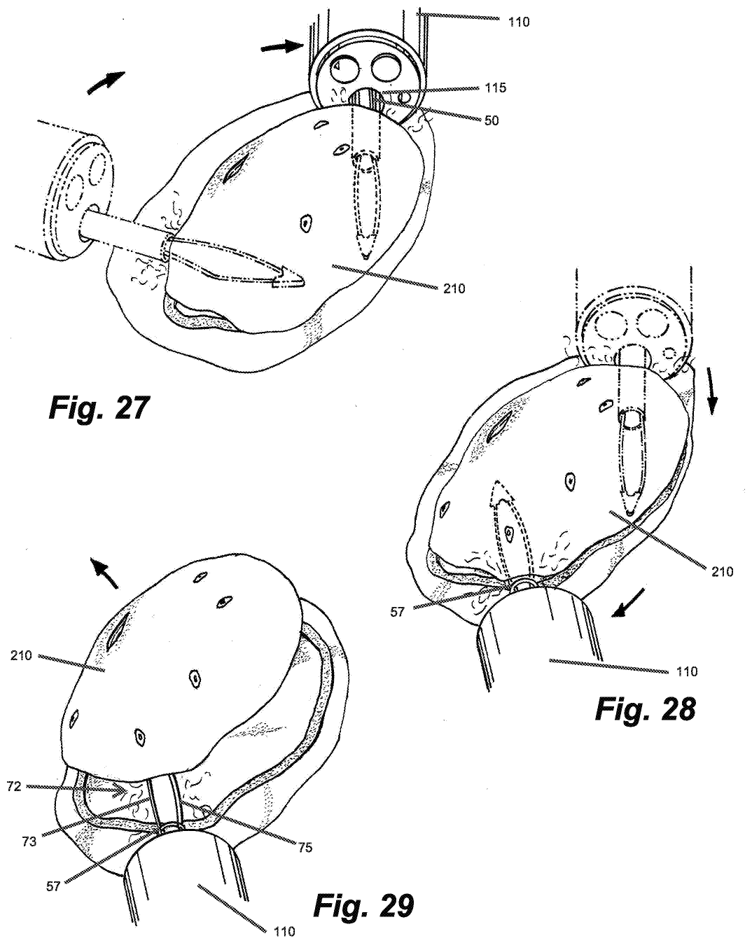

FIG. 26 shows the flexible knife 72 extending a further distance from the distal end 57 of the sheath 50, resulting in the flexible knife 72 being fully deployed from the sheath 50. The extended flexible knife is then arranged at the right side of the lesion or polyp and cuts from right to left to reach the edge of the lesion or polyp 210, as shown in FIG. 27. The endoscope and flexible knife remain parallel to the gastrointestinal wall, and the end of the endoscope is spaced a distance from the flap of the lesion or polyp that has been cut further. In FIG. 28, the flexible knife is shown cutting around the perimeter of the lesion or polyp 210 while remaining parallel with the gastrointestinal wall. Cutting around the perimeter of the lesion or polyp is the final step in removing or freeing the lesion or polyp from the gastrointestinal wall. The freed lesion or polyp 210 may be removed from the patient and subject to further analysis, e.g. diagnosis and treatment recommendations, once the perimeter has been cut, such as shown in FIG. 29.

Various modifications of the invention and many further embodiments thereof, in addition to those shown and described herein will become apparent to those skilled in the art from the full contents of this document. The subject matter herein contains important information, exemplification and guidance that can be adapted to the practice of this invention in its various embodiments and equivalents thereof.

1. A medical instrument comprising:
a proximal handle comprising an electrical connection plug, a slidable handle portion, and a rotatable handle portion;
a sheath extending from the proximal handle, the sheath comprising a lumen;
a wire extending through the sheath, the wire including a working end, the wire and the sheath moveable relative to each other to cover and reveal the working end such that the working end has a retracted position and a working position, the working end comprising an exposed portion of the wire for electrical cutting and an insulated portion of the wire covered by an insulating material; and
an insulated spear-shaped tip disposed over a distal end of the insulated portion of the wire and adjacent to the exposed portion of the wire, the insulated portion of the wire extending through the insulated spear-shaped tip, the insulated spear-shaped tip for preventing cutting of a layer other than a mucosal layer or a submucosal layer.

2. (canceled)

3. The medical instrument of claim 1, wherein the working end comprises a wire loop.

4. The medical instrument of claim 1, wherein the insulating material covers the working end from the insulated spear-shaped tip to the proximal end of the working end.

5. The medical instrument of claim 1, wherein the slidable handle portion is manipulatable by a user to move the working end forward and backward between the retracted position and working position.

6. The medical instrument of claim 1, wherein the rotatable handle portion is manipulatable by the user to rotate the working end.

7. The medical instrument of claim 1, wherein the sheath is operable by the proximal handle to cover and reveal the working end.

8. The medical instrument of claim 1, wherein the wire is operable by the proximal handle to cover and reveal the working end.

9. The medical instrument of claim 8, wherein the proximal handle further comprises graduated markings on a body of the proximal handle corresponding to a length of the wire for extending or retracting to reveal or cover the working end.

10. The medical instrument of claim 1, wherein the wire is insulated.

11. The medical instrument of claim 1, wherein the electrical connection plug is configured to connect to an electric power source.

12. The medical instrument of claim 1, wherein the proximal handle further comprises a stabilizing collar where the sheath extends from the proximal handle, the stabilizing collar for holding by the user when rotating the proximal handle.

13. The medical instrument of claim 1, wherein the proximal handle further comprises a second slidable handle portion.

14. The medical instrument of claim 13, wherein the second slidable handle portion is connected to a metal lumen, the metal lumen extending from the proximal handle in the sheath, the second slidable handle portion moving the metal lumen forward or backward from the proximal handle in the sheath.

15. The medical instrument of claim 13, wherein the proximal handle further comprises a setting clip placed between the slidable handle portion and the second slidable handle portion to prevent forward movement of the slidable handle portion past a position and backward movement of the second slidable handle portion past a second position.

16. The medical instrument of claim 1, wherein the medical instrument is used for an endoscopic mucosal resection (EMR) or an endoscopic submucosal dissection (ESD).

17. The medical instrument of claim 1, wherein the sheath of the medical instrument is configured to operate within a working channel in an endoscope.

18. A method of using the medical instrument of claim 1 comprising:
extending the working end a first distance from the sheath and making an initial incision into a lesion or polyp;
extending the working end a second distance from the sheath and electrically cutting into the lesion or polyp;
extending the working end a third distance from the sheath and electrically cutting deeper into the lesion or polyp; and
cutting around the perimeter of the lesion or polyp for en bloc removal of the lesion or polyp.

19. The method of claim 18, wherein electrically cutting is in a sweeping motion from one side of the lesion or polyp to the opposite side of the lesion or polyp.

20. The method of claim 18, wherein dissection of the lesion or polyp comprises cutting of the mucosal layer or submucosal layer.

21. The method of claim 20, wherein the insulated spear-shaped tip guides the flexible knife into the lesion or polyp and prevents cutting of a layer other than a mucosal layer or a submucosal layer.
